(19) United States
(12) Patent Application Publication
(10) Pub. No.: US 2019/0385726 A1
(43) Pub. Date: Dec. 19, 2019

(54) NETWORKED AUTOMATED SYSTEM FOR MANAGING END-TOEND PILL DISPENSING, PACKING, AND SUPPLY-CHAIN

(71) Applicants: Miteshkumar Ishwarbhai Patel, Sacramento, CA (US); Raj Kalpesh Patel, Sacramento, CA (US)

(72) Inventors: Miteshkumar Ishwarbhai Patel, Sacramento, CA (US); Raj Kalpesh Patel, Sacramento, CA (US)

(73) Assignee: Dosepack LLC, San Juan, PR (US)

(21) Appl. No.: 16/524,160

(22) Filed: Jul. 28, 2019

Related U.S. Application Data
(63) Continuation of application No. 15/704,056, filed on Sep. 14, 2017.

(51) Int. Cl. G16H 20/13; G07F 17/00; G06F 16/2457

(52) U.S. Cl. CPC G16H 20/13; G06F 16/24575; G07F 17/0092

(57) ABSTRACT

An automated system is disclosed for managing end-to-end medical pill dispensing, medical pill packaging, and medical pill inventory process. The system is in a computerized networked environment. The system is used for medical pills in a blister pack in accordance with a prescription that is specific to a patient. The system is also used for dispensing and packaging a small to high volume of blister packs. The system is further used for managing pill inventory. The system is automated through its end-to-end process of pill packaging and delivery.

The system includes a central server. The central server acts as the core decision maker for the operation of the system. Alternate servers or decision making is also contemplated. The server includes a processor, memory, and decision logic module.

The system also includes one or pill dispensing machines, wherein each of the plurality of pill dispensing machines. These pill dispensing machines are communicatively connected with each other and with the central server through a network. The pill dispensing machine includes several pill containers and each pill container houses several medical pills. These containers are able to dispense one pill at a time. The central server or the pill dispensing machine provides instruction the containers on when and which pill to dispense.

The pill dispensing machine includes a hopper and a collection tray. This tray has a grid of slots that match the grid of slots on a blister pack. An enclosed tunnel inside the pill dispenser connects the pill container(s) with the hopper. In process, the hopper acts as the entry way for the pills dispensed to the collection tray and subsequently to the blister pack.

The system includes one or more robot transport vehicles. These are also connected to the central server through the network. They include wheels and move in a path that is directed by the central server. They transport either an empty, filled, or partially filled blister pack from one location/station to another location/station in the system.

The system includes a smart mobile device that is capable of downloading a mobile application. The mobile application communicates with central server and is used for setting system preferences, getting system updates, or providing operational instructions.

The system includes a parking station that has parking spots where the robot transport vehicle can be parked. It is communicatively connected to the central station.

The system includes a printer station and a sealing station. These are used for printing a label that is sealed onto the blister pack.

The system includes a storage station/rack and an unloading station. The completed blister packs are unloaded from the robot car at the unloading station and placed into storage. Alternatively, a gripper is also used to unload.

The printer station, sealing station, unloading station, and storage station/rack are all connected to the central server through the network. These components can also be integrated into the pill dispenser.

If an error occurs in the dispensing or packing process at any of the stages, the error is flagged and the blister pack associated with the error is taken to a manual verification station.

The system is modular and stackable allowing flexibility to expand or contract based on volume and space needs. The system also utilizes decision logic and its logic tree and deep learning processes to improve system operations.

### CONTINUATION INFORMATION

This application is a continuation-in-part of an application Ser. No. 15/710,819, titled “SYSTEM AND METHOD FOR DISPENSING MEDICINE USING A MANUAL FILL TRAY APPARATUS,” having Miteshkumar Ishwarbhai Patel and Raj Kalpesh Patel as inventors, filed on Sep. 20, 2017, which is a continuation in part of application Ser. No. 15/704,056, titles, “MEDICAL DISPENSING SYSTEM WITH FEEDBACK PRE-FILL APPARATUS,” filed on Sep. 14, 2017. Application Ser. Nos. 15/710,819 and 15/704,056 are also incorporated herein by reference.

### FIELD OF THE INVENTION

The present invention relates to a networked automated system for managing end-to-end pill dispensing, packing, and supply-chain. More particularly, the present invention relates to a computerized process that uses a variety of hardware devices that are networked together to manage pill inventory and dispense and package pills in a blister pack for several patients while utilizing routing and efficiency management tools to fill a large quantity of blister packs.

### DISCUSSION OF THE RELATED ART

Pill fulfilment machines and processes are used to dispense pills and package them in a blister pack. Several systems use different components for achieving each step of the process, for example, a system may have partial process of dispensing performed by a machine while the remaining portion of the process manually performed.

In some instances, pills are manually loaded into containers or cartridges to store the pills. The cartridges and the containers are loaded onto machines such that the machine can extract the pills from the cartridge to dispense it. One such system is defined in PCT application bearing International Publication Number WO 94/15859. Such systems are not capable of performing complete automation and lack several necessary steps that are involved in an end-to-end packaging of a pill or blister pack. They do not produce a pill/blister pack as their end product but simply dispense pills in a particular timed order. As such they are rudimentary and require several steps to be performed manually. One such example in the WO 94/15859 patent is the step of manually actuating a dispense bar to eject medications into a tray.

Other attempts have been made at dispensing pills that involve a semi-automated process. One such example includes U.S. Pat. No. 8,434,641 that is used for filling vials with pills. This patent makes a case for a pharmacist to concentrate more on patient interaction and less on pill fulfilment and as such proposes the semi-automate invention that has several manual steps as well. There are several disadvantages of using the machine and method described in U.S. Pat. No. 8,434,641. Here we focus on one major disadvantage that relates to the amount of space required for this machine and process. Pharmacies are typically located in a grocery or retail stores or as part of a bigger store off-the counter medical store (such as Walgreens, CVS, and Rite-Aid), or in a hospital. Regardless of their location, they have space constraints. The invention of U.S. Pat. No. 8,434,641 requires a large amount of space which is not always available in a pharmacy setting. Further, such systems focus only on filling pills in a vial and to do so include several manually actuated levers and gate mechanism to hold a vial in place during fulfilment.

Similar to addressing space constraints, yet another problem with the current systems is that they do not accommodate for expansion or contraction in the system and are not scalable. It's a one-type solution that does not adapt to the various sizes of pharmacies nor does it expand or contract to address the volume fulfilment needs, i.e. higher or lower volumes. Not having the flexibility makes the system either underperform to address its requirements or occupy a larger space when the institution (such as Pharmacy, Clinic, or Hospital) has a lesser fulfilment requirement.

As such, the above-mentioned shortcomings ultimately lead to a laborious manual process or an inefficient process that does not perform end-to-end pill packing in an efficient manner. Therefore, a system that overcomes these disadvantages is required.

### SUMMARY OF THE INVENTION

An automated system is disclosed for managing end-to-end medical pill dispensing with computer vision, pill packaging and its delivery, and medical pill inventory. The system also includes capability to perform pill inventory with future predictions and forecasting. The system also includes capability to pack and sort pills, including automated sorting, and provide verification of pills through computer vision.

The system is in a computerized networked environment. The system is used for dispensing and packaging medical pills in a blister pack in accordance with a prescription that is specific to a patient. The system is also used for dispensing and packaging a small to high volume of blister packs, sorting pills, and verify pills that have been dispensed. The system is further used for managing pill inventory, including various inventory management functions such as supply chain management, forecasting, predicting needs, and ordering pills. The system is automated through its end-to-end process of pill packaging.

The system includes a central server. The central server acts as the core decision maker for the operation of the system. Alternate servers or decision making is also contemplated. The server includes a processor, memory, and decision logic module.

The processor processes all the system commands and instructions and directs the operation of all the components. The processor also accesses a decision logic when needed. The decision logic applies decision tree analysis and deep learning over tie to make decisions that improve the efficiency and performance of the system. The deep learning aspect is performed over time where each system cycle is stored and analyzed for its performance. The subsequent cycle is improved based on system performance, hurdles, time delays, and any system issues observed in the previous performance and used to enhance future cycles. With accumulation of data and each new problem encountered over time, the system continues to learn and improve subsequent cycles.

The system also includes one or more pill dispensing machines, wherein each of the plurality of pill dispensing machines are communicatively connected with each other and with the central server through a network. The pill dispensing machine includes several pill containers and each pill container houses several medical pills. These containers are able to dispense one pill at a time. The central server or the pill dispensing machine provides instruction to the pill containers. These instructions guide the pill container on when and which pill to dispense.

The system includes a pack separator that is used for separating a single blister pack from a stack of blister packs such that the separated blister pack can be transported to a pill dispensing machine for use.

The pill dispensing machine includes a hopper and a collection tray. This tray has a grid of slots that match the grid of slots on a blister pack. An enclosed tunnel inside the pill dispenser connects the pill container(s) with the hopper. In process, the hopper acts as the entry way for the pills dispensed to the collection tray and subsequently to the blister pack. The pill dispensing machine uses a plurality of drawers to house canisters that hold pills as well as a row of drawers that can be manually filled with expensive, delicate, and not often used pills.

The system includes one or more robot transport vehicles. These are also connected to the central server through the network. They include wheels and move in a path that is directed by the central server. They transport either an empty, filled, or partially filled blister pack from one location/station to another location/station in the system. The central server uses deep learning and artificial intelligence concepts to direct the path of the robot transport vehicle and chose the best and most optimized path for the robot transport to reach its destination. Several other uses of deep learning have been described throughout the application.

The system includes a smart mobile device that is capable of downloading a mobile application. The mobile application communicates with central server and is used for setting system preferences, getting system updates, or providing operational instructions.

The system includes a parking station that has parking spots where the robot transport vehicle can be parked. It is communicatively connected to the central station. The parking station communicates with the central server to provide its availability, queue, and any other operational issues.

The system includes a printer station and a sealing station. These are used for printing a label that is sealed onto the blister pack. Both of these communicate with the central server to provide its availability, queue, and any other operational issues.

The system includes a storage station/rack and an unloading station. The completed blister packs are unloaded from the robot car at the unloading station and placed into storage. Alternatively, a gripper is also used to unload.

The printer station, sealing station, unloading station, and storage station/rack are all connected to the central server through the network. These components can also be integrated into the pill dispenser.

If an error occurs in the dispensing or packing process at any of the stages, the error is flagged and the blister pack associated with the error is taken to a manual verification station.

The system is modular and stackable. The modularity allows the flexibility to expand or contract based on volume and space needs.

### BRIEF DESCRIPTION OF THE DRAWINGS

The accompanying drawings are included to provide further understanding of the invention and constitute a part of the specification. The drawings listed below illustrate embodiments of the invention and, together with the description, serve to explain the principles of the invention, as disclosed by the claims and their equivalents.

FIG. 1 illustrates a networked automated system for end-to-end pill inventory management, pill dispensing, and pill packaging according to the disclosed embodiments;

FIG. 2 illustrates components of an unloading station according to the disclosed embodiments;

FIG. 3 is a block diagram that illustrates the process flow of the system according to the disclosed embodiments; and

FIG. 4 illustrates the communications between the robot transport vehicle and the pill dispensing stations according to the disclosed embodiments.

### DETAILED DESCRIPTION OF THE PREFERRED EMBODIMENTS

Aspects of the invention are disclosed in the accompanying description. Alternate embodiments of the present invention and their equivalents are devised without parting from the spirit or scope of the present invention. It should be noted that like elements disclosed below are indicated by like reference numbers in the drawings.

An automated system is disclosed for managing end-to-end medical pill dispensing with computer vision, pill packaging and its delivery, and medical pill inventory. The system also includes capability to perform pill inventory with future predictions and forecasting. The system also includes capability to pack and sort pills, including automated sorting, and provide verification of pills through computer vision.

The system is in a computerized networked environment. The system is used for dispensing and packaging medical pills in a blister pack in accordance with a prescription that is specific to a patient. The system is also used for dispensing and packaging a small to high volume of blister packs, sorting pills, and verify pills that have been dispensed. The system is further used for managing pill inventory, including various inventory management functions such as supply chain management, forecasting, predicting needs, and ordering pills. The system is automated through its end-to-end process of pill packaging.

Further, as mentioned above, the automated and networked systems and their components described herein are directed to pill inventory management, pill dispensing, pill packaging, and pill package storage and delivery. The described inventions provide end-to-end automation for a full cycle, start to finish, pill dispensing and packaging process. and complete start to finish.

The disclosed embodiments include several hardware components that are used to accomplish the full pill dispensing and packaging process. Various decisions and deep learning are deployed through the provided process flow architecture described. Decision logic is utilized in determining the most effective and efficient process path to utilize as well as which hardware components to use to produce maximum amount of pill packages in an accurate manner.

The described systems are modular and stackable that provide for scaling up or down depending on the volume of pill packing desired. The modularity and stackability is also utilized for adjusting to various physical space constraints at the location of the system.

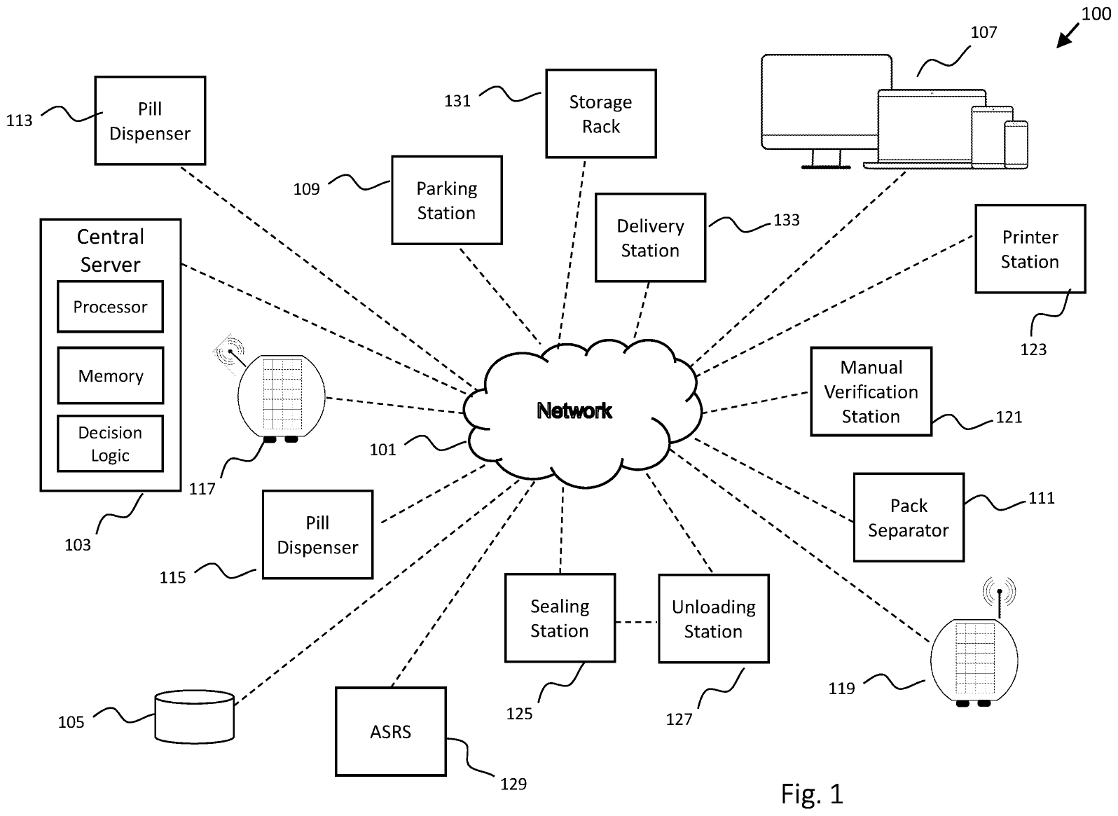

FIG. 1 illustrates a networked automated system for end-to-end pill inventory management, dispensing, packaging, and delivery according to the disclosed embodiments.

The system of FIG. 1 provides a representative environment in which the invention can be implemented. Aspects of the invention described in FIG. 1 includes hardware components as well as some general communications and process instructions or computer-executable instructions, such as routines executed by a general-purpose data processing device, such as, a server computer, laptop or personal desktop computer, and/or a mobile device. Those skilled in the relevant art will appreciate that the invention can be practiced with other communications, data processing, communications, or computer system configurations, including: wireless mobile devices, Internet capable devices, and wearable computers, multi-processor or micro-processor-based systems or programmable consumer electronics and the like. The terms “computer,” “server,” are used interchangeably and may refer to any of the above devices and systems.

Further, although certain functions are described as being performed exclusively on a single hardware device or in a single process, the invention can also be practiced in distributed environments where functions or modules are shared among disparate processing devices and various changes are made to the single process, such as running parallel processes, or multi-process to accomplish a similar result as that described in the invention. Further, if a distributed computing environment is used, various program modules may be located in both local and remote memory storage devices.

System 100 may be located in a pharmacy, hospital, a manufacturing warehouse, a clinic, or other medical services facility. The network of devices depicted is able to include any number of devices and any various devices including, but not limited to, a central server 103 connected to the network 101, a source 105, a plurality of laptop or mobile devices 107, a robot car parking station 109, a pack separator 111, plurality of drug dispensers 113-115, a plurality of robot cars 117-119, a manual verification station 121, a printer station 123, a sealing station 125, an unloading station 127, an ASKS station 129, a storage rack 131, and a delivery station 133.

As mentioned earlier, not all the components are required in all the operations and the components are interchangeable, modular, and stackable to fit both the space and volume requirements for pill dispensing and packaging.

Central Server (103)

The central server 103 includes a processor, memory, and decision logic. The one or more processors assists in processing information and instructions for operation of the system 100. The processor, or microprocessor, is coupled to memory, which can be non-transitory storage medium, for executing program instructions. The processor is also configured to provide instructions to various components of the system and those instructions result execution of the operational instructions results in coordination between the components of the system to perform automatic and autonomous end-to-end pill dispensing and packing.

One example of the processor's operation is to obtain patient prescription data from the source 105. Then storing this data into the memory of the central server. Also obtaining the inventory of pills to fulfil the prescription from a plurality of pill dispensing machines. Further obtaining availability of one or more robot cars for transporting a blister package to a desired pill dispensing machine. Storing all the data obtained into the memory. Communicatively working with decision logic to analyze the stored data and process instructions for a particular robot car 117 to travel to a particular dispensing machine for filling the prescription. In performing it tasks, the processor also interacts and works with decision logic on an as needed basis. This is just one example of the processor's function; the example has been simplified to provide a glimpse into its operations. Several other operations and functionality that is described in the figures below is also executed by the processor.

The central server connects to all the components of the system, i.e., the source device 105, the plurality of laptop or mobile devices 107, the robot car parking station 109, the pack separator 111, plurality of drug dispensers 113-115, the plurality of robot cars 117-119, the manual verification station 121, the printer station 123, the sealing station 125, the unloading station 127, the ASRS station 129, the storage rack 131, and the delivery station 133, and uses its processor, memory, and decision logic to analyze and perform operations on the entire system thereby acting as a central command. The central server's database includes a list of patient information, prescription information, medication information, and other information needed to dispense and package pills into a blister pack, as well as component details, such as capability, availability, scheduling etc. Alternatively, the central server 103 obtains some of the unavailable data through the source 105. As mentioned earlier, the connectivity with the components allows the central server 103 to provide instructions to all the components such that they can communicate with each other and operate in accordance with the optimal plan of operation.

Decision Logic

The decision logic is part of the central server 103. Decision logic or decision logic device or means is responsive to the processor, also part of the central server 103, and is used to automatically engage in a decision-making process. Once data on operation of the system, including operation, availability, and inventory of each component in the system is accessed and stored into the memory, the decision logic takes over to find the best path, the most efficient process, or the best utilization of each component and provides the decision logic for the processor to process and execute commands resulting in the actual functionality and operation of the system.

In one example, the decision logic may determine the number of cars parked in a parking station and decide to send an unmarked robot car to a separate parking station if it is efficient to navigate to the separate parking station in a shorter amount of time. It may also access traffic data stored in the memory to suggest the best path for the robot car 117. Path determination, system operational efficiency, supply chain management, component utilization is just a few of the processes that are analyzed by the decision logic, several other processes, some of which are described in further detail below are also contemplated.

To accomplish its decision, such as a decision involving path determination, system operational efficiency, supply chain management, component utilization etc., the decision logic means compares the data obtained by the processor and stored in the memory with certain threshold values or a counter. The threshold values and counter may be pre-determined or the decision logic may constantly update the threshold or the counter based on historical data and deep learning to refine the model and suggest a new threshold or counter. These thresholds or counters can be used to determine path determination, system operational efficiency, supply chain management, component utilization.

For example, a threshold may be to allow a maximum of 3 robot cars on a particular path or to a certain parking station, if the new robot car enters the system, and the threshold is reached, the decision logic may provide alternative path towards parking stations. The decision logic may also keep track of the time periods during which each component is available and operational as well as its capacity, for example, to determine utilization, underutilization or over utilization.

The decision logic applies decision tree analysis and deep learning over tie to make decisions that improve the efficiency and performance of the system. The deep learning aspect is performed over time where each system cycle is stored and analyzed for its performance. The subsequent cycle is improved based on system performance, hurdles, time delays, and any system issues observed in the previous performance and used to enhance future cycles. With each cycles of data and each new problem encountered, the system continues to learn and improve subsequent cycles.

Client Computing Devices (107)

The system includes connectivity to a plurality of client devices 107, such as laptops, desktops, tablets, or other mobile devices. These devices 107 may include at least one other client application that is configured to receive information and content, such as patient information, medication information, or dispensing and packaging data, from another computing device or from the central server 103.

The client application may include a capability to provide and receive textual content, or multimedia information, such as patient information, medication information, or dispensing and packaging data. The client application may further provide information that identifies itself, including a type, capability, name, or the similar identifying information. They may also uniquely identify themselves using phone number, mobile identification number (MIN), an electronic serial number (ESN), mobile device identifier, network address, such as IP (Internet Protocol) address, media access control (MAC) layer identifier.

The client computing devices 107 may also be configured to communicate a message, such as through email, short message service (SMS), or multimedia message service (MMS), to the central server or another computing device.

One or more of the devices is able to be an end user, a company and/or another entity. In some embodiments, peer-to-peer sourcing is implemented. For example, the source of the data to be compared with is not on a localized source but is found on peer sources.

Source (105)

As mentioned earlier, system 100 also includes a source 105. The central server 103 is communicatively coupled to the source through the network 101. The source or source device 105 can be any device that contains a source such as a searchable database, web pages, medical records, EMR (electronic medical records), patient prescription data, physician notes, patient medical history, or any other information or device that provides information relating to patient, patient's medicines, prescriptions, medication side effects, or dispensing, filling, and packaging of medications. The source device is also capable of accessing the Internet.

The network 101 can be any network or number of networks such as the Internet, cloud networks, an intranet, Ethernet, a LAN/WAN, either wired or wireless, satellite, a combination of any of the mentioned networks that allow communications.

Drug Dispenser

The pill dispenser 115, also referred to as pill dispensing robot, drug dispenser, or pill dispensing machine includes various compartments or storage locations for storing pills and customized medications. These pill storage areas can be a canister, a manual fill tray, or other storage boxes. The canister is a storage box with a rotating mechanism that dispenses a pill upon rotation of the internal mechanism. Several drawers, in varied sizes and shapes, both large and small can store a plurality of canisters in each drawer, for example one drawer can store 16 canisters. Several storage areas, canister drawers, and plurality canisters are located in the pill dispenser 155 and together they can store over 256 varieties of pills and medications. Additional pills are also stored in manual fill trays.

The dispensing section of the pill dispenser includes a hopper, which is a funnel shaped conduit through which pills are dropped into a tray. The tray, also known as pre-fill tray or collection tray, have a grid of slots that mimic the grid of slots in a blister pack. The grid of slots represents the days of the week and the times of the day. Once pills are collecting in the tray, after they have been dropped through the hopper, the collected pills are then transferred to a blister pack. The blister pack is located in the robot car, such as robot car 117 or 119, that has been assigned to deliver the empty blister pack to the dispensing machine and collect the filled blister pack for transporting it to another station in the system 100.

The pill dispenser 115 may also include other components such as packing plate, which acts as a second collection plate after the pre-fill tray, an additional hopper, which may be smaller than the hopper, and other components that allow dispensing and collection of pills to be ultimately transferred to a blister pack. The dispensing robot also has its own processor and memory. The processor communicates with Central Server 103 through network 101, Source 105 through network 101 or through Central Sever 103. The dispensing machine through its processor obtains the prescription of a particular patient and saves it in its memory. It uses this patient prescription data to dispense pills from selected canisters, manual fill trays, and canister drawers that correspond with the pills for the prescription. The processor intercuts the appropriate canister, manual fill tray, or other storage areas in the pill dispenser 115 that hold the corresponding pill to be dispensed and dropped into the hopper to be delivered to the pre-fill tray and ultimately in the appropriate slot into the blister pack. Since the blister pack slots correspond with days of the week and times of the day, each pill is designated for a specific slot such that the pill is to be taken on a certain day and certain time of the day that corresponds with the hours of administration (HOA) that is in the patient prescription.

In another embodiment, the pill dispenser receives all the dispensing commands and instructions from a mobile application or directly from the central server 115. It then dispenses the pills as instructed for a specific patient and populates the slots of the blister pack. Other methods of instructing the pill dispenser 115 are also contemplated.

Although details of one individual pill/drug dispenser has been described, the network is not so limited and includes a plurality of pill dispensers. The number of pill dispensers in a system 100 depends on several factors. These include volume needs, space requirements, churn rate to pack a certain number of blister packages per day, the type of facility, e.g., large hospital or pharmacy to a smaller facility or clinic or nursing home. The number may also depend on amount and variety of medication that need to be stored and dispensed. For example, a large hospital that has a high patient turn over rate and a large number of patients in their facility may requires a faster and higher volume of blister packs. If the hospital has the space, since it has a high requirement to package a large amount of pill packs, it is likely that multiple pill dispenser will be part the system 100. Likewise, a smaller facility, such as a smaller clinic or nursing home or pharmacy that has lesser requirements and a lower capacity to fill a lower number of blister packs will likely have just 1 or lesser pill dispenser.

Robot Transport Parking Station (109)

The robot car parking station 109 is a modular station where multiple robot cars 117-119 can be parked. It is essentially an electro mechanical structure that is used for parking robot cars. The parking station can have several floors and one robot car 117 can fit on each floor. The parking station may also be designed to accommodate more than one car on each floor. The number of floors can vary and the parking station is modular such that floors can be added or removed as desired or required by the system 100. In yet another example, there will be a robot car parked on a single floor.

The number of robot car parking slots, floors, and number of robot cars parked on each floor also depends on the total number of robot cars used by system 100 as well as the need to address a certain volume of pill dispensing. For example, a large system that has a high volume, high capacity, and a high requirement to package a large amount of pill packs and includes several pill dispensing machines 115 will likely have a higher number of robot cars 117-119 and likely a higher number of robot car parking stations, which results in a larger number of robot car parking slots within a station as opposed to a smaller system that has lesser requirements and a lower capacity to fill a lower number of blister packs.

The parking station may be a transitory stop for the robot car 117-119 or it may be housed and parked there for a longer term when it is not being used. The system 100 through its central server 103 keeps track of all robot cars and where they are parked at any given time. As such, the system 100 is aware whether to send a robot car to a particular parking station based on the parking station capacity, reservation, or availability.

Pack Separator (111)

A pack separator 111 is device that includes a mechanism that separates one blister pack from another. Since blister packs are stacked on top of each other it becomes necessary to separate and obtain a single blister pack for filling the medications for a single patient. The separator functions to separate a single pack and then have it loaded onto the robot car 117 for transporting it to its next destination. To do so, the pack separator uses a plurality of sensors. These sensors are capable of finding the separation lines between the stacked blister packs. A mechanical arm then separates the packs and places an empty blister pack onto the robot car.

Plurality of Robot Transports 117-119

The robot car 117, also shown at 119, and also referred to as robot transport or dose car or robot vehicle transport, is a mobile car that can transport a blister pack from one location to another location. It is guided and directed by the central server 103. It may also be guided and directed by other means, such a decision logic of the central server 103, or another database or mobile application that can track the movement of components in the system 100.

The robot car 117 travels in tracks. The tracks allow movement in a straight path. It also includes several stop points. The stop points are where the robot car 117 switches its XY direction. The robot cars 117 can also be lifted to a higher level and transferred from one track to another.

The robot car 117 includes wheels and certain electronics that allow it to navigate from one location to another. It also includes either an antenna, transmitter, or other type of communication module that can receive and send signals and receive instructions that guide its path of travel.

Manual Verification Station (121)

The manual verification station 121 is a mechanical structure designed for the manual intervention. This station may or may not be used depending on its need. In one embodiment, the manual verification station is used as an error correction step in the pill dispensing and packing process. It can also be used as a secondary check or verification of the pills already populated into a blister pack.

In one possible use, it is required when an error is detected by the pill dispenser 115 during its dispensing process. The error may be that a wrong pill has been populated in the wrong slot of the blister pack or that pill from an unintended canister was dropped that does not match the prescription for the patient.

The manual verification station is communicatively connected to the pill dispenser 115 through network 101. It is also communicatively connected to the central server 103 through network 101. Aside from the errors mentioned above, other errors that are detected in dispensing or filling that are either detected by the pill dispenser 115 or reported to the central server 103, such as for example, missing pills from a certain cell of the blister pack, can also be flagged for inspection and then transported to the manual verification station 121. A particular blister pack that is flagged is transported to the manual verification station either manually or by the robot car 117. At this station, each cell of the blister pack is verified and checked in accordance with the patient prescription to ensure that all the pills are correctly populated in the appropriate times slots for the patient.

Printer Station (123)

The printer station 123 is a mechanical structure that is used for printing labels that are placed on the blister packs. The printer station is communicatively connected to the pill dispenser 115 through network 101. It is also communicatively connected to the central server 103 through network 101.

The labels printed by the printer station include patient data and prescription data. The patient data can be patient name, address, phone number or other patient related information. The pill data can be hours of administration, medication information, side effects of the medication, and other information that may be helpful to the patient in administering the medication. It can also include details for taking the medication before or after a meal.

Although a single printer station 123 is disclosed in system 100, the system may include several printer stations that are situated in various locations. In one embodiment, the printer station is physically located next to or very near a pill dispenser. This is to facilitate the printing of the labels and placing them on the blister pack as a next step after the blister pack has been filled by the pill dispenser. In operation, a robot car 117 may carry the filled blister pack from the pill dispenser 115 to the printer station 115 upon receiving instruction from the central server 103 or its decision logic module.

The number of printer stations 123 in a system depend on the amount of printing and labeling needed, i.e. the amount of blister packs to be filled. It also depends on the need to address a certain volume of pill dispensing, i.e., a large system such as a hospital system that has a high volume, high capacity, and a high requirement to package a large amount of pill packs and includes several pill dispensing machines 115 will likely have a higher number of printer stations as oppose to a smaller system that has lesser requirements and a lower capacity to fill a lower number of blister packs will have just one or fewer printer stations.

Sealing Station (125)

The sealing station 125 is a mechanical structure designed for sealing the label that was previously printed by the printer station 123. To do so the sealing station applies a calculated amount of heat on the label and the heat causes the label to stick to the blister pack. Since the label contains glue at its bottom, upon application of the heat, the glue melts or liquifies and bods the label with the blister pack thereby resulting in the labels sticking to the blister pack.

Although a sealing station 125 is disclosed in system 100, the system may include several sealing stations. As mentioned above, this is dependent on the need, volume, and churn rate or blister packs to be prepared in a day or within a certain time frame. As such larger facilities, such as hospitals, that has a high volume, high capacity, and a high requirement to package a large amount of pill packs will also include a higher number of sealing stations in their system 100.

In operation, the sealing step comes after the label is printed by the printer station. As such, it makes logical flow to have the sealing station located nearby to the printing station. However, the sealing station can be situated in various locations.

In operation, a robot car 117 may carry the filled blister pack from the pill dispenser 115 to the printer station 115. Once a label has been placed on the blister pack, the robot car 117 may then proceed to the sealing station to have the label sealed.

Since the sealing station is also communicatively connected to the pill dispenser 115, central server 103, and printer station 123 through network 101, it can receive commands from the central server 115 that can keep it apprised of the process of the packaging and when to expect the robot car 117 to arrive at the sealing station.

Unloading Station (127)

The unloading station 127 is a station where a completed blister pack gets unloaded from the robot car 117. The unloading station is a two-axis system consists of a vertical axis and a horizontal axis. It uses a pick-and-drop mechanism and to pick up the blister pack from the robot car 117 and place it for storage or next step. In operation, the robot car 117 transports a completed pill pack that has all the pills required for a particular patient along with a heat-sealed label to the unloading station. The pill or blister pack which sits on or within the robot car 117 is then picked up at this unloading station.

System 100 may include one or more unloading stations 127. They may be located in various locations or near a delivery station. The number of unloading stations, as explained with other components, depends on the amount of volume, churn rate, and type of facility. Typically, a facility may have just one unloading station, however, large warehousing or distribution operations may include more.

The unloading station 127 is communicatively connected to central server 103 through network 101. It sends data back to the central server 103 that allows the central server 103 to keep track of the number of packaged units or blister packs completed and delivered or shipped. This helps in inventory management and also to access the amount of completion and the amount left to be completed for the day or for the time duration.

ASRS Station 129

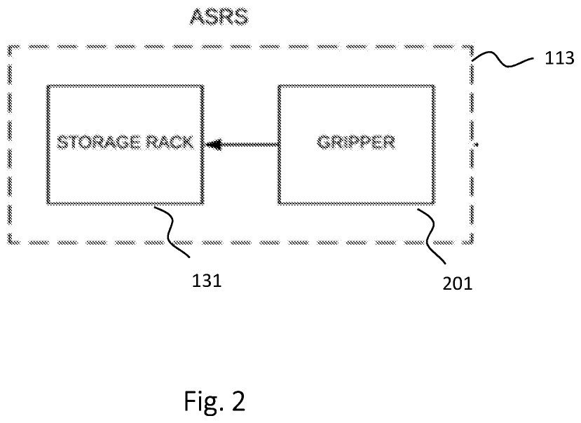

The ASRS station 129 includes a conveyor and a gripper. It is basically a mechanism for loading storage racks and storing blister packs into the storage racks. The ASRS station can be an independent mechanism or it can be connected communicatively through the network to the central server 103 and also to the other components in the system 100. The system may include multiple ASRS stations. Additional detail of the ASRS and Gripper are described in FIG. 2.

Storage Rack 131

Once the blister pack is unloaded at the unloading station 127, it is then placed in storage at the storage rack 131. The storage rack 131 may include a plurality of moving mechanisms that assist in placing the completed blister pack in storage. It may also include several floors and layers that allow the blister pack to be stored at various locations. It also includes location tracking through RFID means that is used by central server to track the location of the storage rack. Similar to other components, there may be one or more storage rack that are connected through the network 100 to the central server 103.

The storage rack is a wheeled device that can be transported from one location to another by maneuver it on its wheels. In one embodiment, the storage rack has several rack levels, where each rack level has 8 slots or 8 storage positions. For example, the storage rack, along with its storage slots together may allow the storage of 96 blister packs. Alternatively, it could be more or less depending on the need and the structure of the parking station.

Delivery Station 133

The delivery station 133 may be optional. In some instances, the final blister packs loaded onto the storage racks may be the end of the process and in other instances a delivery station 133 maybe included. The delivery station 133 basically monitors all the blister packages that are being sent out of the facility or send out of the dispensing and packaging station to their intended recipients. It can also be used as a final check step to ensure that pill packages have been properly packaged and have the corresponding label on them.

FIG. 2 illustrates components of an unloading station according to the disclosed embodiments. In one embodiment, the ASRS station is integrated and part of the unloading station 113. The ASRS station includes a storage rack 131 and a gripper 201. The storage rack may be the same storage rack as mentioned earlier in or a separate storage rack that acts as an interim storage rack to hold blister packs until they are transferred to the final or portable storage rack.

The gripper 201 is a mechanical structure that gathers a completed or filled blister pack from the unloading station. It is a box like cavity that can hold blister pack and it is capable of rotating its axis 90 degrees so that it carries blister pack from conveyor and takes it to storage rack for final delivery. It is designed in a way that it holds blister pack securely until it reaches storage location. Once it reaches the specified location, pusher mechanism inside box like cavity pushes blister pack to storage location.

In operation, the gripper 201 communicates with the central server to perform its movements and gather the blister pack. Once the central server indicates that a completed blister pack is ready for pick up from the unloading station, then the gripper 201 gathers the specified blister pack and moves it to a free space in the storage rack.

Modularity & Stacking

Referring back to FIG. 1, the central server 103, plurality of laptop or mobile devices 107, a robot car parking station 109, pack separator 111, plurality of drug dispensers 113-115, plurality of robot transports 117-119, manual verification station 121, printer station 123, sealing station 125, unloading station 127, ASRS station 129, storage rack 131, and delivery station 133 together form the components of the system 100. These components are also referred to as system hardware components or hardware components in this application.

In one embodiment, the system may not include pack separator 111, ASRS station 129, and delivery station 133. Alternatively, these three components may be included, however, may not be communicatively coupled to the other hardware components through network 101, thereby operating in a separate individual and an as needed capacity.

In another embodiment, the hardware components are modular and stackable. For example, a plurality of parking stations may be stacked on top of each other or next to each other to form a larger capacity parking station that has double the number of parking spaces as a single parking station. Likewise, several hardware components, such as printer station, sealing station, pill dispensers can also be stacked together, either vertically or horizontally, to accommodate for a higher and larger capacity.

The system is also modular in the sense that various components can be added, integrated, or removed based on the capacity needs.

In one example, the pill dispensing machine includes a plurality of canisters that are housed in a plurality of canister drawers, Based on the volume needs, the number of canisters or drawers can be added or removed in the pill dispensing machine,

Yet another example of modularity is the number of floors in a parking station. If a particular parking station has four floors and five are needed, the system can be modified to add an additional floor to allow for additional robot cars to be parked on the additional floor. Likewise, if the system has a lesser need and a lesser number of robot cars, the one or more floors of the parking station can be removed to reduce the footprint and space occupied by the parking station. Although a few examples have been discussed, the entire system is modular and stackable.

Integration into Pill Dispenser

In one embodiment, the printer station 123 is integrated into the housing of the pill dispenser 113. The printing station 123, or when integrated—the printer, may be placed towards the lower portion of the pill dispensing housing and in closer proximity to the blister pack. In operation, once all the pills intended for a particular blister pack have been dispensed by the canisters and they made their way through several compartments of the pill dispenser, such as the hopper and collection tray, ultimately to the blister pack, then the blister pack is complete. At this point the pill dispenser may direct the printer to print a label that is to be placed on the blister pack.

Likewise, in another embodiment, the sealing station 125 is integrated into the housing of the pill dispenser 113. The integrated sealing station 125 is used for sealing the label that has been placed on the completed blister pack by the printer. The sealing station may apply heat onto the label to melt the glue that is place on its bottom such that the bottom of the label sticks to the blister pack.

If an integration approach is used, both the printer and the sealer (sealing station) become part of the pill dispensing unit and may not need to communicate separately with the central server. Instead, the pill dispenser as a whole with all its components, the printer and sealer becoming one of its components if integrated, shall communicate and provide printer and sealer updates, availability, and queue to the central server 103.

Similarly, the storage rack or storage space may also be integrated into the housing of the pill dispenser 113. The integrated storage space may be used for housing and storing completed blister packs. The storage area may have stacking capability to stack and store the completed blister packs that can then be removed either manually or through the robot transport vehicle 117.

RFID

As mentioned above, one object of the invention is to transport an empty blister pack, get the blister pack filled with designated pills from the pill dispenser, and then transport the blister pack to different stations within the system, 100, such as printer station, sealing station, and unloading station, to name a few, to deliver a completed pack for sending it to the intended patient. To do carry the blister pack through these process steps, a robot vehicle transport 117 is used.

The blister pack that is used through the process includes a radio frequency identification (RFID) tag. The RFID tags contain electronically stored information that is transmitted it from the central server 103. This information is patient specific and includes patient details such as patient name, birth date, types of prescriptions and pills designated for the patient and other patient data relevant and needed by the system 100 to operate.

The RFID tag ensures that only the medicines/pills designated for a specific patient are populated in the correct blister/pill pack. Since system 100 may have several, or even hundreds of blister packs being transported, dispensed, and packaged, the RFID provide a layer of assurance to avoid any errors, such as mislabeling the wrong blister pack, or populating it with pills that are intended for another blister pack that is for another patient.

All the components, and the robot vehicle transport 117, verify the RFID tag before performing needed steps or operations onto the blister pack, such as filling it, labeling it, or sealing it. Several RFID readers are displaced across the system and in some instances each component may have their own RFID reader to check and verify the RFD on the blister pack before proceeding.

Processing & Instructions

Some of the functions of the processor, which is located in the central server 103, were mentioned earlier. It is to be noted that the central server may include more than one processor and may delegate separate functionality to each of the processors.

The processor can execute, access, generate, and/or modify the functionality of the components mentioned above. The processor may use decision logic in regulating the operations and functionality. More specifically, the processor may execute programs that include routines, objects, components, data structures, and the like that perform particular tasks or implement particular abstract data types.

Once a command/program is executed by the processor, a device or component in the network, such as the robot vehicle transport 117 will act in accordance with the instructions provided. For example, the processor, based on guidance from the deep learning algorithm from the decision logic, may determine that a particular path is usually busy or a certain component takes a certain amount of time to complete its process, as such, the processor may instruct, and the robot vehicle transport 117 may follow a designated path that is not the shortest path to the next destination.

Although not shown, the central server 103 may also include a communication infrastructure, such as a communication bus, that is coupled with the processor.

Reservations

As mentioned above, system 100 components such as pill dispenser, printer station, sealing station, unloading station, as well as robot vehicle transports communicate with the central server. One key aspect is that they provide status update, availability, and queue. Having this knowledge of each component, the central server is also able to maximize output by reserving components for certain time slots and amount of duration. As such, it has planning ability to map out the entire operation and plan ahead before the start or the process or improvise and change during the process. The deep learning aspect, performed by the decision logic, obtains all the system data as well as reservation data to improve subsequent cycles.

Mobile Application

In one embodiment, a mobile device 107 is communicatively connected to the central server 103 through network 101. The mobile device 107 can also be connected to other components of the system through network 101 and communicate with them through central server 103.

The mobile device may be mobile phone, a smart watch, a tablet, or a laptop. Mobile device may also be an independent portable and mobile console or device that is capable of connecting to the Internet and downloading a mobile application.

The system 100 may include or more mobile devices. Each mobile device comprises memory configured to store computer-readable instructions such as applications and other programs, and a processor configured to execute the applications and other programs stored in memory. These programs can be routines, objects, components, or data structures that perform particular tasks or implement particular abstract data types. The mobile device may also include other logic components to perform the various tasks in the course of managing the provision of applications to mobile devices. For example, such instructions may be executable to communicate with central server 103 over network 101 to communicate, select preferences, or get an update.

The mobile device shall have the capability of downloading a mobile application. The mobile device may connect to the central server and use the mobile application to display various system operations and functionality. Using the mobile device's downloaded mobile application, a user can set preferences, provide instructions, receive system updates, such as details of the pill dispensing process, availability of any component in the system 100, inventory of pills in the system 100, queue for any component or the entire system, or other inventory, status, and operational updates. The user may be able to make changes to the operations and functions of the system and may also be able to turn ON or OFF a particular component of the system 100 or the entire system 100 in case of an emergency.

The mobile application may be secured and need authentication to allow a user to login using a password and provide certain rights to different types of user. For example, a manager of person involved with management of the process may have all the rights to make process changes while others that simply need an update may only have reading and viewing rights.

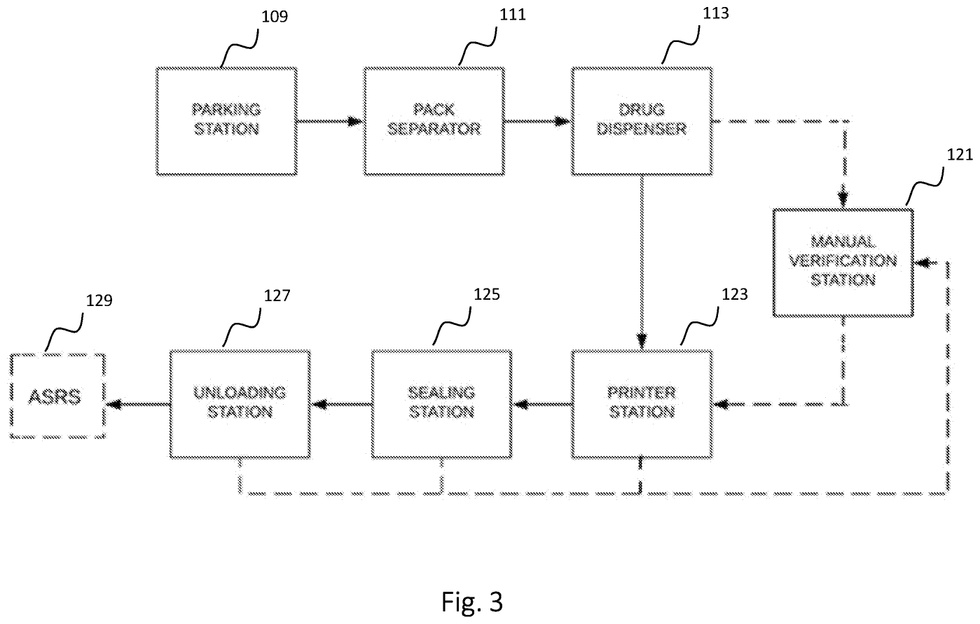

FIG. 3 is a block diagram that illustrates the process flow of the system according to the disclosed embodiments. System 100 includes hardware operational components that function to dispense pill and package them into a blister pack and monitor system inventory and optimization thereby resulting in an efficient and high-volume pill package producing system.

A subset of these components is used to physically dispense and package the blister pack and deliver it. These include a parking station 109, pack separator 111, plurality of drug dispensers 113-115, a manual verification station 121, a printer station 123, a sealing station 125, an unloading station 127, an ASKS station 129, which includes a storage rack 131 and a gripper. The transportation between these subsets of components/stations is performed by a plurality of robot cars 117-119.

In one embodiment, the solid lines between each station, also referred to as the basic path, is performed. In an alternative embodiment, additional steps that are marked by dotted or broken lines are also performed on an as needed basis. The flow and transport starting from paring station 109 and ending at either the unloading station 127 or ASRS 129 is performed by one or more robot vehicle transports 117-119.

The start of the process is at the parking station 109 where the robot vehicle transport 117 may be parked. While parked, the robot vehicle transport 117 may also be getting charged or powered up for it to transport blister packs. The charging while being parked avoids downtime for charging.

Next, the robot vehicle transport 117 proceeds to the pack separator 111 to obtain a blister pack. After obtaining the blister pack, the robot vehicle transport 117 then proceeds to the pill dispenser where the blister pack is filled with pills in one or more slots of the blister pack.

Upon completion, the robot vehicle transport 117 proceeds to the printer station where a label with patient information and pill information is placed on the completed blister pack.

Alternatively, if there is an error during dispensing or some other error that requires verification or error correction, the robot vehicle transport 117 proceeds to manual verification station before going to the printer station.

After the printer has placed a label in the blister pack, the robot vehicle transport 117 then proceeds to the sealing station 125. At this stage a heating application is applied to the label to release the glue on the label and have the label stick to the blister pack.

Thereafter, the robot car 117 transports the completed and sealed blister pack to an unloading station 127. A pneumatic mechanism at this station unloads the blister pack from the robot vehicle transport 117 and places onto a conveyor. Gripper 201 picks the blister pack from the conveyor and places it at a selected position or node in the storage rack.

If an error is detected at the printer station, sealing station, or unloading, the robot vehicle transport 117 takes the blister pack to the manual verification station where error correction is performed.

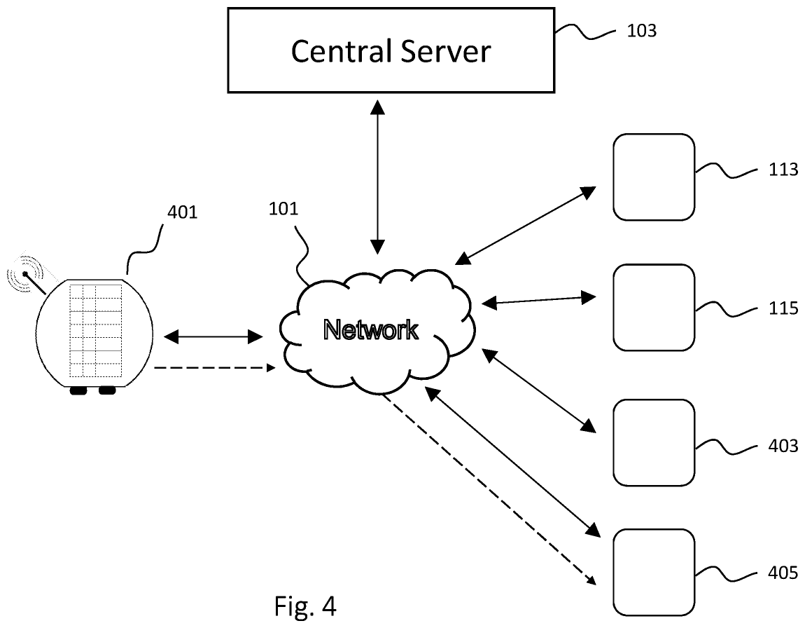

FIG. 4 illustrates the communications between the robot transport vehicle and the pill dispensing stations according to the disclosed embodiments. In this embodiment, four pill dispensers 113, 115, 403, and 405 are depicted. This figure illustrates one possible decision-making process performed by decision logic of the central server 103.

In this embodiment, the decision logic pings each pill dispensing machine 113, 115, 403, and 405 to obtain their status, availability, and queue. It also asks for data relating to any functional problems with the pill dispensing machine.

Also depicted is a robot car 401 that is awaiting instructions to proceed. The decision logic also monitors the traffic flow of all the robot cars. The decision logic also accesses the prescription to be filled for the blister pack that is currently placed on robot car 401 and determines which of the four pill dispensers contain al the pills to fulfil that specific prescription.

Based on all the criteria mentioned above the decision logic comes to a decision for providing an optimal path for the robot car 401. It then sends the decisional information after having parsed through a logic decision tree to the processor of the central server 103. In return, the processor executes commands that instruct the robot car 401 to proceed to pill dispenser 405. The processor communicates the command through network 101. The robot car 401 receives the processors instructions and follows the path directed to go to pill dispenser 405.

Although one exemplary path and process stage of travelling to the pill dispenser 405 is described, the process is similar for proceeding between multiple components and stations. For example, alternative processes in which alternatives travel paths are used by the robot cars are also contemplated.

It will be apparent to those skilled in the art that various modifications and variations may be made in the disclosed embodiments of the disclosed methods and systems without departing from the spirit or scope of the invention. Thus, it is intended that the present invention covers the modifications and variations of the embodiments disclosed above provided that the modifications and variations come within the scope of any claims and their equivalents.

What is claimed is:

1. An automated system for managing end-to-end medical pill dispensing, medical pill packaging, and medical pill inventory in a computerized networked environment, wherein the medical pills are being packaged in a blister pack for a patient in accordance with a prescription that is specific to the patient, the system comprising:
a central server, wherein the central server includes a processor, memory, and decision logic module,
a plurality of pill dispensing machines, wherein each of the plurality of pill dispensing machines are communicatively connected with each other and with the central server through a network, wherein each pill dispensing machine includes a plurality of pill containers, wherein each pill container includes a plurality of pills and is able to dispense one pill at a time upon receiving an instruction to do so from either the pill dispensing machine of the central server, wherein each pill dispensing machine includes a hopper and a collection tray having a plurality of slots, wherein an enclosed tunnel connects one or more pill containers with the hopper, wherein the hopper places the pills dispensed from the pill containers into selected slots of the collection tray, wherein the slots of the collection tray mimic the slots of a blister pack and the collection tray pills are dispensed into the blister pack when the dispensing process is completed;
a plurality of robot transport vehicles, wherein each of the plurality of robot transport vehicles are communicatively connected with each other and with the central server through a network, wherein the robot transport vehicles received instructions from the central server to follow a path and either receive, store, or transport the uncompleted or completed blister pack from one location to another location;
a smart mobile device having a display screen, wherein the smart mobile device is communicatively connected to the central server through the network, and the smart mobile device having downloaded a mobile application for interacting with the central server, wherein the central server, wherein the smart mobile device is capable of receiving system information from the central server and processing the received information using the mobile application to display it on the display screen of the smart mobile device;
a parking station having a plurality of parking spots, wherein the parking station is communicatively connected to the central server through the network, wherein the parking station is communicatively connected to the robot transport vehicle, wherein the parking station, when pinged by either the robot transport vehicle or the central server, responds by providing its parking availability for all of its parking spots; and
the central server, plurality of dispensing machines, plurality of robot transport vehicles, the smart mobile device, and parkin station, which form the components of the system, communicate with each other over the network and obtain data and instructions from the central server's processor, wherein the memory stores computer-readable program instructions, and the processor coupled to the memory executes the program instructions to provide operational instructions to each of the components of the system, wherein execution of the operational instructions results in coordination between the components of the system to maximize the number of blister packs that can be packaged.

2. The automated system of claim 1, further comprising a printer station.

3. The automated system of claim 2, wherein the printer station is integrated into and is part of the pill dispensing machine.

4. The automated system of claim 2, wherein the printer station is an independent station that is communicatively connected with the pill dispensing machine and the central server through the network to be recognized as a component of the system, wherein the printing station prints a label that is to be placed on a blister pack having a plurality of medical pills, wherein the label includes specific information, wherein the information is specific to the medical pills that are packaged in the blister pack.

5. The automated system of claim 4, wherein the system includes a plurality of printer stations, wherein each printer station is communicatively connected with the central server through the network, wherein each printer station updates the central server of its availability to print a label and its current printing queue.

6. The automated system of claim 1, further comprising a sealing station.

7. The automated system of claim 6, wherein the sealing station is integrated into and is part of the pill dispensing machine.

8. The automated system of claim 6, wherein the sealing station is an independent station that is communicatively connected with the pill dispensing machine and the central server through the network to be recognized as a component of the system, wherein the sealing station seals a completed blister pack that includes a plurality of medical pills, wherein the sealing station applies a heat seal to a label placed on the blister pack thereby forming a sealed bond between the label and the blister pack.

9. The automated system of claim 8, wherein the system includes a plurality of sealing stations, wherein each sealing station is communicatively connected with the central server through the network, wherein each sealing station updates the central server of its availability to seal a blister pack and its current sealing queue.

10. The automated system of claim 1, further comprising a storage station.

11. The automated system of claim 10, wherein the storage station is integrated into and is part of the pill dispensing machine and is used for storing completed blister packs.

12. The automated system of claim 10, wherein the storage station is an independent station that is communicatively connected with the central server through the network to be recognized as a component of the system, wherein the storage station is portable and includes wheels that allow its transport from one location to another, wherein the storage station includes one or more floors with each floor having one or more storage spaces to store a completed blister pack.

13. The automated system of claim 12, wherein the system includes a plurality of storage stations, wherein each storage station is communicatively connected with the central server through the network, wherein each storage station updates the central server of the number of storage spaces available and number of storage spaces filled.

14. The automated system of claim 1, further comprising an unloading station, wherein the unloading station picks a completed blister pack from the robot transport vehicle and places the completed pack onto a conveyor, which is then transported to a storage station.

15. The automated system of claim 14, wherein the unloading station includes a gripper, wherein the unloading station uses the gripper to grip the blister pack from the conveyor and place it in the storage station.

16. The automated system of claim 1, wherein the robot transport vehicle carries an empty blister pack to a designated dispensing station.

17. The automated system of claim 1, wherein the robot transport vehicle transports a completed blister pack from the dispensing machine to a printer station, wherein the printing station prints a label and places it on the completed blister pack.

18. The automated system of claim 17, wherein the robot transport vehicle transports the completed blister pack from printer station to a sealing station for sealing the label onto the blister pack.

19. The automated system of claim 1, further comprising a manual verification station, wherein the manual verification station is used for manually verifying the contents of the blister pack, wherein the robot transport vehicle transports the blister pack from the pill dispensing machine to the manual verification station if the blister pack has been flagged for an error either by the pill dispensing machine of the central server.

20. The automated system of claim 1, further comprising a blister pack separator, wherein the blister pack separator includes mechanical means for separating a single blister pack from a plurality of blister packs stacked on top of each other, wherein the blister pack separator places the separated blister pack on the robot transport vehicle thereby allowing its transport to the dispensing machine.
